(19) United States
(12) Patent Application Publication
(10) Pub. No.: US 2014/0243578 A1
(43) Pub. Date: Aug. 28, 2014

(54) RADIOWAVE TREATMENT FOR CANCER

(71) Applicant: Radiowave Therapy Research Institute Pty Ltd, Claremont (AU)

(72) Inventor: John Alfred Gorton HOLT, Nedlands (AU)

(73) Assignee: Radiowave Therapy Research Institute Pty Ltd, Claremont (AU)

(21) Appl. No.: 14/182,484

(22) Filed: Feb. 18, 2014

Related U.S. Application Data
(60) Provisional application No. 61/768,896, filed on Feb. 25, 2013.

(51) Int. Cl. A61N 5/10

(52) U.S. Cl. CPC A61N 5/1031
USPC 600/1

(57) ABSTRACT

A method of treating cancer in an adult subject comprising an electromagnetic (EM) radiation treatment step of administering to the subject an effective dose of EM radiation of a frequency in the range of 430-440 MHz (UHF) within a period of ≦45 minutes, the EM radiation being provided by a UHF emitter defining an exposure region, wherein for the duration of the period the subject is reclined upon a horizontal or substantially horizontal surface, and wherein the surface and UHF emitter are arranged for relative movement such that the exposure region passes, continually or periodically, between a first position and at least a second position on the subject. The method may further comprise an auxiliary treatment step such as X-ray therapy or treatment with certain chemical agents such as oxidised glutathione (GSSG) and/or a precursor thereof (eg glutathione (GSH)) in combination with a non-toxic oxidising agent, and S-methyl-L-cysteine sulphoxide (SMCO)

### TECHNICAL FIELD

The present invention relates to methods for cancer treatment.

### BACKGROUND

The use of ultra-high frequency (UHF) microwave electromagnetic (EM) radiation in human cancer therapy is known. For example, it is known that when a patient is exposed to such radiation prior to X-ray therapy, the cancer cell kill can be increased by between about three and one hundred times compared with X-ray therapy alone. Further, it has been previously reported (see Australian Patent Specification No 643156) that if, at the time of administering the UHF radiation, an effective amount of one or more selected chemical agents is provided to or in the vicinity of the cancer cells, there may be no need to subsequently administer X-ray therapy; that is, the X-ray therapy may be avoided while still achieving at least satisfactory levels of cancer cell kill. The chemical agents that have been previously reported as being suitable in such a treatment include non-toxic organic disulfides (eg oxidised glutathione (GSSG)) and/or a precursor thereof (eg glutathione (GSH) in combination with a non-toxic oxidising agent), and non-toxic organic sulfoximines (eg methionine sulfoximine).

It has been previously considered that UHF radiation may act to enhance the effect of a cancer treatment such as X-ray therapy or a treatment with the chemical agents described above. Moreover, it has been previously considered that such UHF radiation may also enhance the effect of other treatments involving, for example, chemotherapy with cytotoxic agents such as taxanes (eg paclitaxel), antineoplastics (eg doxorubicin and epirubicin), topoisomerase inhibitors (eg etoposide and irinotecan) and vinca alkaloids (eg vincristine and vinblastine), and y radiotherapy. The present invention relates to the identification of particular modes and features for applying a UHF radiation step in a method of treating cancer, which can result in enhanced cancer cell kill and/or other advantages.

### SUMMARY

In a first aspect, the present invention provides a method of treating cancer in an adult subject, said method comprising an electromagnetic (EM) radiation treatment step of:

administering to the subject an effective dose of EM radiation of a frequency in the range of 430-440 MHz (UHF) within a period of ≦45 minutes, said EM radiation being provided by a UHF emitter defining an exposure region, wherein for the duration of said period the subject is reclined upon a horizontal or substantially horizontal surface, and wherein said surface and UHF emitter are arranged for relative movement such that the exposure region passes, continually or periodically, between a first position and at least a second position on said subject.

Preferably, the surface and UHF emitter are arranged for relative linear movement such that the exposure region passes, continually or periodically, back and forth between a first position and at least a second position on said subject at a rate of 5-15 feet per minute. The UHF emitter may be an array of two or more emitters, and positioned within 0.5 to 3 inches of the subject.

In a second aspect, the present invention provides a method of treating cancer in an adult subject, said method comprising:

an electromagnetic (EM) radiation treatment step of administering to the subject an effective dose of EM radiation of a frequency in the range of 430-440 MHz (UHF) within a period of ≦45 minutes, said EM radiation being provided by a UHF emitter defining an exposure region, wherein for the duration of said period the subject is reclined upon a horizontal or substantially horizontal surface, and wherein said surface and UHF emitter are arranged for relative movement such that the exposure region passes, continually or periodically, between a first position and at least a second position on said subject; and an auxiliary treatment step or steps of
(a) infusing said subject with
   a dose of 3-10 g of glutathione (GSH), wherein said GSH is formulated in combination with at least one suitable oxidising agent, and/or a dose of 3-10 g of oxidised glutathione (GSSG), provided that where the subject is infused with both GSH and GSSG, the total dose of the GSH and GSSG is no more than 10 g, and
   a dose of 3-10 g of S-methyl-L-cysteine sulphoxide (SMCO), wherein said SMCO is formulated in combination with at least one suitable oxidising agent, and/or
(b) administering to said subject an effective dose of X-ray radiation.

### BRIEF DESCRIPTION OF FIGURES

FIG. 1 provides graphical results for real-time cell proliferation of MO59K glioblastoma cells and control (ie non-cancerous) human retinal cells (ARPE), following radiowave exposure (434 MHz) with or without pre-treatment with oxidised glutathione (GSSG);

FIG. 2 provides a bar graph of relative cell counts of human prostate cancer cells (PC-3) and control (ie non-cancerous) human prostate cells (PNT1A) following pre-treatment with various concentrations of a GMI™ preparation comprising glutathione (GSH) and S-methyl cysteine sulphoxide (SMCO) and five daily UHF (434 MHz) exposures: CTRL=no UHF exposure, EXP=UHF exposure only, (a) 0.1×, (b) 0.04×, (c) 0.016×, (d) 0.0064×, (e) 0.00256× GMI;

FIG. 3 provides a bar graph showing the results of in vitro assays of a first embodiment of a cancer treatment according to the present invention on human prostate cancer cells (PC-3) and control (ie non-cancerous) human prostate cells (PNT1A): relative cell counts before and after treatments (CTRL=no UHF exposure);

FIG. 4 provides a bar graph showing the results of in vitro assays of a first embodiment of a cancer treatment according to the present invention on human prostate cancer cells (PC-3) and control (ie non-cancerous) human prostate cells (PNT1A): viability of cells following the cancer treatment;

FIG. 5 provides a bar graph showing the results of in vitro assays of a second embodiment of a cancer treatment according to the present invention on human prostate cancer cells (PC-3) and control (ie non-cancerous) human prostate cells (PNT1A): relative cell counts before and after treatments; and

FIG. 6 provides a bar graph showing the results of in vitro assays of a second embodiment of a cancer treatment according to the present invention on human prostate cancer cells (PC-3) and control (ie non-cancerous) human prostate cells (PNT1A): viability of cells following the cancer treatment.

### DETAILED DESCRIPTION

The present invention relates to the identification of particular modes and features for applying a UHF radiation step in a method of treating cancer.

Thus, in a first aspect, the present invention provides a method of treating cancer in an adult subject, said method comprising an electromagnetic (EM) radiation treatment step of:

administering to the subject an effective dose of EM radiation of a frequency in the range of 430-440 MHz (UHF) within a period of ≦45 minutes, said EM radiation being provided by a UHF emitter defining an exposure region, wherein for the duration of said period the subject is reclined upon a horizontal or substantially horizontal surface, and wherein said surface and UHF emitter are arranged for relative movement such that the exposure region passes, continually or periodically, between a first position and at least a second position on said subject.

The method is broadly applicable to cancer in an adult subject, but is particularly suitable for the treatment of a cancer selected from the group consisting of a malignant neoplasm of the epithelium (for example, a carcinoma such as squamous cell carcinoma, basal cell carcinoma, transitional cell carcinoma, adenocarcinoma or cystadenocarcinoma), pleural and/or peritoneal surfaces (for example, mesothelioma of those surfaces), pigment cells (for example, a malignant melanoma), neural or brain tissue (for example, neurofibrosarcoma, malignant glioma, medulloblastoma or neuroblastoma), kidney tissue (for example, a nephroblastoma), eye tissue (for example, a retinoblastoma), connective tissue (for example, a sarcoma or meningioma such as myxosarcoma, fibrosarcoma, liposarcoma, Kaposi's sarcoma, chondrosarcoma, osteosarcoma, myosarcoma, leiomyosarcoma, rhabdomyo-sarcoma, angiosarcoma, haemangiosarcoma, lymphangiosarcoma or malignant meningioma), lymphoreticular and haematopoietic tissue (for example, a malignant non-Hodgkin's lymphoma, lymphocytic leukaemia, plasmacytoma and multiple myeloma, reticulum cell sarcoma, Hodgkin's disease, granulocytic leukaemia or monocytic leukaemia) and embryonic-originating tissue (for example, a malignant teratoma, teratocarcinoma or chorioncarcinoma).

A UHF emitter or an array of two or more such emitters may be used to provide the effective amount of the UHF radiation. The frequency of the UHF radiation will be in the range of 430-440 MHz, preferably in the range of 432-436 MHz and, more preferably, in the range of 433.5-435.0 MHz (eg about 434 MHz). The UHF emitter(s) may comprise one or more microwave antennae. The, or each, antenna may comprise a single circular turn approximately 19 cm in diameter (the diameter being chosen in order that it resonates at the frequency of the transmitter to create minimum reflected power) arranged such that the plane of the circular antenna is held substantially tangential to the curvature of the subject's body, the subject being reclined upon a horizontal or substantially horizontal surface. Such a circular antenna may emit predominantly H-wave electromagnetic radiation of wavelength between about 25 and 30 inches (most preferably about 27 inches) towards the subject's body. While the presence of E-wave radiation generated by dipole antennae (folded or of other configuration) will not necessarily hinder the efficacy of the UHF radiation, it can lead to greater heating of the subject's skin and a poorer depth dose than a circular or loop antenna. Since such effects are clearly undesirable, dipole antennae are therefore less preferred. Each antenna may be suitably connected to a UHF generator by a coaxial cable. All equipment within about 2 metres of the antennae should be of non-metallic materials such as wood or plastic, including, for example, the horizontal or substantially horizontal surface upon which the subject reclines. The antennae themselves may, preferably, be housed in plastic enclosures (eg of polypropylene), with about 14 inches clearance between adjacent enclosures.

The, or each, antenna will preferably be within 0.5 to 3 inches of the subject's skin. Where an array of two or more antennae (eg 3 or 4 antennae) is used, it may be preferable to arrange the antennae around the subject's body, thereby defining an exposure region that encircles the subject. The total antennae power should preferably be less than about 6 kW (eg adjustable between about 2 kW and about 4 kW for a 4 antennae array) in order to remain tolerable to the average subject; with each antenna providing power in the range of 500 W to 1500 W. Each antenna within an array may emit UHF radiation of slightly different frequency (eg in a 4 antennae array, there may be four different frequencies such as 434.0 MHz, 434.1 Mhz, 434.5 MHz and 434.7 MHz) so as to avoid the generation of standing waves.

The surface (upon which the subject is reclined, generally lying on his/her back but sometimes on his/her side, during the period of administration of the UHF radiation) and the UHF emitter(s) are arranged for relative movement such that the exposure region passes, continually or periodically, between a first position and at least a second position on said subject. Preferably, said surface and UHF emitter(s) are arranged for relative linear movement such that the exposure region passes, continually or periodically, back and forth between a first position and at least a second position on said subject at a rate of 5-15 feet per minute, more preferably at a rate of about 10 feet per minute. Moreover, preferably, the UHF emitter(s) is/are in a fixed position and said surface is moved back and forth. Thus, in some embodiments, where an array of two or more UHF emitters is used, the emitters effectively define a channel through which the subject can pass, reclined on a horizontal or substantially horizontal surface on a movable support.

The first and second positions effectively define the region of the subject upon which UHF radiation is administered. That is, for a localised administration (eg in a treatment of cancer at a defined site), the first and second positions will be as required to ensure that the exposure region passes across the desired site (eg in the case of breast cancer, one or both breasts) and, preferably, no other part of the subject's body. Otherwise, the first and second positions will be as required to ensure that the exposure region passes across a more extensive part of the subject's body (eg the head, neck and torso) or indeed, the whole or substantial whole of the subject's body (eg the knee to vertex). Whole body exposure is clearly desirable where systemic cancers such as leukaemias are concerned and/or where there is a need to treat known or possible metastases of the cancer. In some embodiments, the method of the present invention may comprise a combination of localised UHF administration and administration of UHF radiation to a more extensive part of the subject's body or, more preferably, the whole or substantial whole of the subject's body. In such embodiments, the exposure region may pass, continually or periodically, between (a) a first position and a second position on said subject defining a part of the body for localised administration of UHF radiation, and (b) a third position and fourth position on said subject defining a more extensive part of the body or, otherwise, the whole or substantial whole of the body, for administration of UHF radiation. The administration of UHF radiation per (a) and (b) may be performed in either order.

The effective dose of the UHF radiation is administered to the subject within a period of ≦45 minutes.

The period of administration of UHF radiation may comprise two to four exposure stages (ie wherein UHF radiation is administered) separated by rest stages (ie wherein no UHF radiation is emitted). The total time of the exposure stages is preferably no more than 25 minutes and, more preferably, is of about 10 to 22 minutes. For example, in one embodiment of the method, the effective dosage of the UHF radiation is administered within a period of 30 minutes, wherein the period comprises three exposure stages of 9 minutes, 9 minutes and 4 minutes duration (ie a total of 22 minutes). Further, the two initial 9 minute exposure stages may each consist of a localised UHF administration while the third, 4 minute exposure, consists of administration of UHF radiation to a more extensive part of the subject's body or, more preferably, the whole or substantial whole of the subject's body.

The method of the first aspect may further comprise an auxiliary treatment step such as X-ray therapy or treatment with certain chemical agents such as oxidised glutathione (GSSG) and/or a precursor thereof (eg glutathione (GSH)) in combination with a non-toxic oxidising agent, and S-methyl-L-cysteine sulphoxide (SMCO).

Thus, in a second aspect, the present invention provides a method of treating cancer in an adult subject, said method comprising:

an electromagnetic (EM) radiation treatment step of administering to the subject an effective dose of EM radiation of a frequency in the range of 430-440 MHz (UHF) within a period of ≦45 minutes, said EM radiation being provided by a UHF emitter defining an exposure region, wherein for the duration of said period the subject is reclined upon a horizontal or substantially horizontal surface, and wherein said surface and UHF emitter are arranged for relative movement such that the exposure region passes, continually or periodically, between a first position and at least a second position on said subject; and

an auxiliary treatment step or steps of
(a) infusing said subject with
   a dose of 3-10 g of glutathione (GSH), wherein said GSH is formulated in combination with at least one suitable oxidising agent, and/or a dose of 3-10 g of oxidised glutathione (GSSG), provided that where the subject is infused with both GSH and GSSG, the total dose of the GSH and GSSG is no more than 10 g, and
   a dose of 3-10 g of S-methyl-L-cysteine sulphoxide (SMCO), wherein said SMCO is formulated in combination with at least one suitable oxidising agent, and/or
(b) administering to said subject an effective dose of X-ray radiation.

In the method of the second aspect, the EM radiation treatment step may be conducted as described above in respect of the method of the first aspect. As such, the EM radiation treatment step may employ one or more of the preferred features and/or embodiments described above.

The auxiliary treatment step may comprise infusing the subject with a dose of GSH (and/or GSSG), together with a dose of SMCO, and/or by administering a dose of X-ray radiation in accordance with protocols well known to the person skilled in the art (but typically at lower doses of 140-160 rads per day). A particularly preferred X-ray administration protocol for use in the method of the second aspect comprises a treatment program that runs over 4-5 weeks or more (eg 8 weeks), with one session on each weekday (or each day) during the program and such that, during each week, the subject receives at least three sessions (eg 5 sessions given by one session per each weekday, or 7 sessions given by one session per each day) of UHF exposure followed by X-ray administration (eg about 10 to 30 minutes of X-ray exposure), for a total of up to 15 UHF/X-ray sessions or more. Optionally, the auxiliary treatment may additionally comprise up to 10 X-ray only sessions per subject over the duration of the treatment program.

Where X-ray is used, it is preferred that the EM radiation treatment step precedes the X-ray administration. Moreover, it is preferred that the period of administration of EM radiation is of 10 to 20 minutes, and that the X-ray administration be commenced within about 20 minutes of the EM radiation treatment step.

Where an auxiliary treatment step of infusing a dose of GSH (and/or GSSG) with SMCO is used, it is preferred that the EM radiation treatment step follows the infusion; for example, it is particularly preferred that the EM radiation treatment step is completed within 30 minutes of the infusion of the GSH (and/or GSSG) with SMCO. As such, the effective dose of electromagnetic radiation administered in the EM radiation treatment step may be provided during two to four, preferably three, periods within the 30 minutes.

The auxiliary treatment step preferably comprises infusing the subject with a dose of about 5 g of GSSG and a dose of about 5 g of SMCO. The exact doses, however, may vary depending upon a variety of factors including the body mass index (BMI), age, general health, sex and diet of the particular subject. In particular, the exact doses of GSSG and SMCO may vary depending upon the subject's BMI.

Where the auxiliary treatment step comprises infusing the subject with GSH, the GSH is formulated in combination with at least one suitable oxidising agent. Suitable oxidising agents are preferably selected from non-toxic organic peroxides and hydroperoxides. A particularly preferred oxidising agent is cumene hydroperoxide, although any of the hydroperoxides of cumene may be used. T-butyl hydroperoxide is another particularly preferred oxidising agent for use in the present invention.

The SMCO is also formulated in combination with at least one suitable oxidising agent. Again, suitable oxidising agents are preferably selected from non-toxic organic peroxides and hydroperoxides, such as cumene hydroperoxide and t-butyl hydroperoxide.

The oxidising agent(s) may be used at a concentration of up to about 1.5 g/l (eg from about 0.25 to 1 g/l, preferably about 0.4 g/l).

The expression “non-toxic” used herein refers to an acceptably low level of toxicity when a compound is present at an effective amount, and not necessarily to a complete absence of toxic effects. In particular, compounds which have only a temporary intoxicating effect and do no significant permanent harm will be referred to and understood as “non-toxic” herein.

Preferably, the method of the second aspect is performed in a manner whereby the auxiliary treatment step and the EM radiation treatment step are both repeated on at least 9 separate days thereafter within 3 weeks of commencement of the method (ie the auxiliary treatment step and the EM radiation treatment step are both repeated on at least 9 separate days after day one). More preferably, the auxiliary treatment step and the EM radiation treatment step are both repeated on 15 separate days within 3 weeks of commencement of the method.

In one embodiment of the method of the second aspect, the method comprises, first infusing a dose of GSH (and/or GSSG) with SMCO, followed by a step of administering the EM radiation and, then, administering a dose of X-ray.

A prime contraindication to the use of the method of the first or second aspect is the presence of large collections of fluid in the pleural or peritoneal cavities. Static collections of fluid which contain high concentrations of salts have heat generated in them by the application of the EM radiation. Subjects with pleural or peritoneal effusions have blockage of the lymphatics draining these areas and also involvement of the pleural and peritoneal membranes. Unless these cavities are dry (ie less than 100 ml fluid when treated), the heating effect can aggravate the irritation of the pleural and peritoneal cavities and the effusions become worse. Recommendations for treatment of such subjects are as follows:
A: Peritoneal effusions. A Le Veen shunt or equivalent should be inserted prior to treatment and must be working so that the peritoneal cavity is kept dry. This drains the ascitic fluid back into the circulation.
B: Pleural effusions. A similar shunt can be used but is much less satisfactory. Pleural paracentesis to drain the cavity together with introduction of some agent causing sclerosis of the pleural cavity which obliterates it is more effective. The introduction of talc, tetracycline or another sclerosing agent appears the best way of preventing failure in the treatment.

Another contraindication is where cancer of the stomach or bowel is causing partial obstruction to the gut. Treatment can cause breakdown of some of the cancer with a perforation of the bowel above the obstruction. All patients with bowel cancer should be fully assessed by a surgeon and if there is any evidence of obstruction, the primary cancer should be removed or exteriorised by colostomy or some other similar procedure. This avoids the likelihood of a perforation, which can be lethal.

In order that the nature of the present invention may be more clearly understood, preferred forms thereof will now be described with reference to the following non-limiting examples.

### EXAMPLES

### Example 1

### Agent Formulations

Agent formulations used in the following Example 2 (where it is referred to as a GMI™ preparation) were prepared according to the following process:

CuOH

Sterile cumene hydroperoxide (CuOH; 80% cumene hydroperoxide) is filtered through 0.2 μm Millex® FG teflon filters (Millipore Corporation, Billerica, Mass., United States of America) into sterile 30 ml vials.

GSH solution
1. A glutathione (GSH) solution is prepared by weighing 25 g of reduced glutathione into a 250 ml sterile container, and sufficient sodium chloride (0.9% w/v) solution added with mixing to produce 250 ml of GSH solution.
2. The GSH solution is then filtered through a 0.8 μm Pall prefilter (Pall Corporation, Port Washington, N.Y., United States of America) with a 0.2 μm Pall sterilising filter fitted with a sterile 19 G Terumo hypodermic needle emptying into a sterile 250 ml intravenous (IV) bag.
3. To each bag, 1.25 ml of the sterile and filtered CuOH is added along with 3.75 ml of sterile t-butyl hydroperoxide (BuOH).

SMCO Solution
1. An S-methyl-L-cysteine sulphoxide sulphoxide (SMCO) solution is prepared by weighing 25 g into a 250 ml sterile container, and then adding sodium chloride (0.9% w/v) solution with mixing to produce 250 ml of SMCO solution.
2. The SMCO solution is then filtered through a 0.8 μm Pall prefilter with a 0.2 μm Pall sterilising filter fitted with a sterile 19 G Terumo hypodermic needle emptying into a sterile 250 ml intravenous (IV) bag.
3. To each bag, 1.25 ml of the sterile and filtered CuOH is added along with 3.75 ml of sterile t-butyl hydroperoxide (BuOH).

### Example 2

### Treatments and Outcomes

This example provides the case history of a number of subjects treated with a method according to the present invention.

### Materials and Methods

GMI Treatment

The GMI™ preparation was administered to the subject by sequentially infusing (via a PICC line) approximately 40 ml of each of the GSH solution and SMCO solution at an infusion rate of about 275 ml/hour.

Radiowave Exposure

Within 30 minutes of the completion of the infusion of the GMI™ preparation, subjects had been given two to three periods of exposure to UHF radiation (434 MHz) as detailed in each case history description.

### Results and Discussion

Case History 1

51 year old male at diagnosis. Diagnosis—Malignant melanoma of left scapula in February 2004. Lesion removed and biopsies showed no metastatic disease to lymph nodes. In March 2006, lesion removed from left chest wall. CT scan showed metastatic disease to left lung and prominent lymphadenopathy (paratracheal node measured 2.0×1.5 cm). Patient commenced treatment in accordance with the invention in July 2006: UHF radiation protocol: 2×10 mins, total antennae power=2 kW (on average). Post-treatment scans showed an initial decrease in size of lung lesion and lymphadenopathy (paratracheal node now measuring 1.3×0.8 cm) and, subsequently, maintained stable disease. A second treatment commenced in April 2007. UHF protocol: 2×10 mins, total antennae power=4 kW. All post-treatment scans showed no convincing evidence of metastatic disease (particularly no evidence of lung lesion). A third treatment commenced in June 2009. UHF protocol: 2×8 mins, 1×5 mins, total antennae power=4 kW. Post-treatment scan showed no evidence of new disease.

Case History 2

38 year old female at diagnosis. Diagnosis—Ductal carcinoma and ductal carcinoma in situ of right breast in June 2007. Mastectomy of right breast with right axillary clearance was performed in July 2007. CT and bone scans showed no evidence of metastatic disease. Patient commenced treatment in accordance with the invention in August 2007. UHF protocol: 2×7 mins, 1×6 mins, total antennae power=3200 W. One year post treatment PET scan showed no recurrence or metastatic disease. Ultrasound of breast and pelvis in June 2011 showed no evidence of disease. This patient's treatment was considered to be systemic; thus the treatment in accordance with the invention was, in this case, follow up treatment post-surgery (akin to the use of chemotherapy and radiotherapy) so as to target possible residual disease.

Case History 3

61 year old male at diagnosis. Diagnosis—Prostate adenocarcinoma, Gleason score 7 (focally 9) in September 2009. Prostate was biopsied and PSA in the same month was 15.7 (N<4.5). In October 2009, MRI showed a focus of abnormal signal in the left peripheral zone of the prostate measuring approximately 1.2×1 cm. There was no spread outside the prostate. Patient commenced treatment in accordance with the invention in October 2009. UHF protocol: 2×8 mins, 1×5 mins, total antennae power=4 kW. Post-treatment in January 2010 PSA was 16.4 and MRI showed decrease in size of prostate lesion to 1.2×0.8 cm. A second treatment commenced in February 2010. UHF protocol: 2×8 mins, 1×5 mins, total antennae power=4 kW. In May 2010, MRI showed a further decrease in prostate lesion to 0.8×0.8 cm. PSA was recorded at 19 and decreased to 17.3. A third treatment was then commenced in July 2010. UHF protocol: 2×8 mins, 1×5 mins, total antennae power=4 kW. In September 2010, PSA was 16.8 and MRI showed stable disease (no change in size of lesion). A fourth treatment commenced in October 2010. UHF protocol: 2×8 mins, 1×5 mins, total antennae power=4 kW. Post-treatment MRI showed stable disease and PSA was 19. Finally, a fifth treatment commenced in March 2011. UHF protocol: Initially 2×8 mins, 1×5 mins, then altered to 1×10 mins, 1×9 mins, 1×3 mins, total antennae power=4 kW. In June 2011, PSA was 13.74 and MRI showed stable disease.

Case History 4

52 year old female at diagnosis. Diagnosis—Colon adenocarcinoma in November 2001. Patient underwent left hemicolectomy followed by five months of chemotherapy. In October/November 2006, CT and PET scans identified a 1.4 cm left para-aortic lymph node mass adjacent to left iliac artery. This was confirmed by biopsy as metastatic disease. CT scan in February 2007 confirmed mass was still present. Patient commenced treatment in accordance with the invention in April 2007. UHF protocol: 2×10 mins, total antennae power=3200 W. CT scan in June 2007 showed reduction in size and definition of lesion, however follow up CT in September 2007 indicated that lymph node mass was still present, but unchanged. A second treatment commenced in November 2007. UHF protocol: 2×7 mins, 1×6 mins, total antennae power=3200 W initially, then altered to 4 kW. Post-treatment CT scans showed no changes in lymph node mass, however CT scans in April and October 2009 and July 2010 found no evidence of metastatic disease.

Case History 5

58 year old male at diagnosis. Diagnosis—Clear cell renal cell carcinoma in March 2006. Patient underwent left upper pole partial nephrectomy. Staging two months later by CT scan showed mediastinal and right hilar lymphadenopathy (largest measuring 2 cm), multiple small nodules in through both lungs (largest 1.3 cm) and a sub-capsular liver lesion (1.9×1.4 cm). Patient commenced treatment in accordance with the invention in July 2006. UHF protocol: 2×10 mins, total antennae power=2 kW (on average). In October 2006, CT scan showed reduction in size of lung lesions (all measuring less than 1 cm), significant lymph nodes now measured only 1.5 to 1.8 cm and the liver lesion was noted again. Two years and five months post-treatment, CT scan described tiny lung nodules (largest measuring 0.4 cm) and no liver metastases were seen. No other treatments were undertaken post-treatment in accordance with the invention.

In all cases, treatment(s) in accordance with the invention were well tolerated with an absence of one or more of the typical side effects of standard chemotherapy or γ radiotherapy (eg hair loss and significant nausea).

### Example 3

### Evaluation of Treatments (I)

Experimentation was undertaken in the laboratory to evaluate the treatment of cancer (ie in accordance with the present invention) using a cancer cell line model. In this example, an investigation was undertaken on the effect of UHF exposure on cell proliferation, with and without treatment with oxidised glutathione (GSSG).

### Materials and Methods

Cells

This example used the MO59-K radiation-resistant glioblastoma cell line, and human non-cancerous retinal ARPE cells (ATCC: CRL-2302™) as a control.

GSSG Treatment

Cells were treated with a 0.05 mM of oxidised glutathione (GSSG), approximately five minutes before UHF exposure.

Radiowave Exposure

The cells, in a 5 mm deep medium, were given a single treatment with 434 MHz (400 W) emitted from a loop antenna at a distance of 50 mm. The single treatment comprised 2×10 minute exposures (1 minute off). The cells were maintained at 1% oxygen and cultured for about five days after the exposures.

Analysis

The effects of the treatment were assessed using a real-time cell proliferation assay (xCELLigence System; Roche Diagnostics Inc, Basel, Switzerland) as per the manufacturer's instructions.

### Results and Discussion

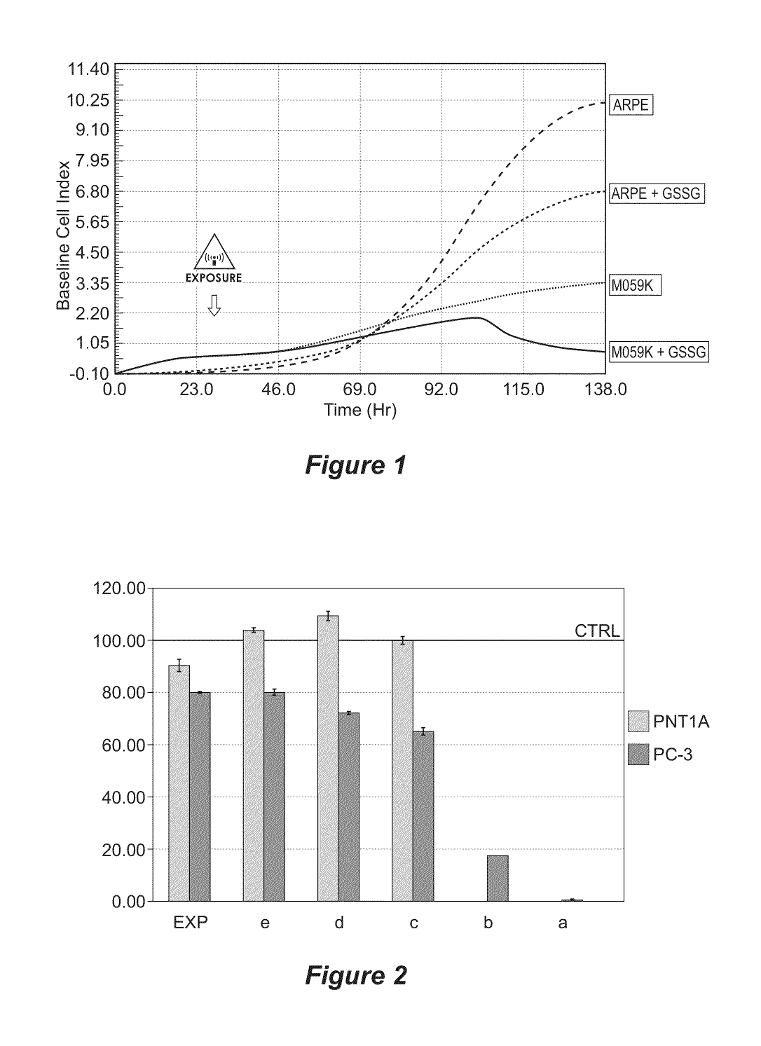

The results are shown in FIG. 1. It is clear that the MO59K cells treated with UHF (434 MHz) in the presence of GSSG begin to die approximately 80 hours post-exposure, while the same cells treated with UHF in the absence of GSSG continued to grow as normal. The control ARPE cells exposed to UHF appeared to grow much faster after the exposure, an effect that was somewhat reduced by the pre-treatment with GSSG. In a related experiment using these cell types (as well as PC-3 and PNT1A), wherein the cells were maintained at 4% oxygen (ie normoxia) instead of 1% oxygen (hypoxia), it was found that the cells respond similarly at both oxygen levels, thereby indicating that the sensitivity of cancer cells to UHF exposure is not hypoxia-dependent.

### Example 4

### Evaluation of Treatments (II)

### Materials and Methods

Cells

This example used the PC-3 human prostate cancer cell line (ATCC: CRL-1435™), and human immortalised prostate PNT1A cells (ECACC: 95012614) as a control.

GMI Treatment

Cells were treated with a GMI™ preparations of various concentrations: (a) 0.1×, (b) 0.04×, (c) 0.016×, (d) 0.0064×, and (e) 0.00256× GMI. For cells that were also subjected to UHF radiation, the cells were treated with the GMI™ preparation approximately five minutes before the exposure to the UHF radiation commenced.

Radiowave Exposure

The cells, in a 20 mm deep medium, were given five daily radiowave treatments with 434 MHz (400 W) at a distance of 50 mm. Each daily treatment comprised 2×10 minute exposures (1 minute off). The cells were maintained at 1% oxygen and cultured for two days after the final exposure (ie 7 days after the first).

Analysis

The effects of the treatment were assessed by routine cell counting.

### Results and Discussion

The results are shown in FIG. 2. It is clear that the higher concentrations of GMI™ are more potent in terms of causing cancer cell death. At the 0.016× GMI™ concentration, it was found that the treatment resulted in significant cell death of the cancerous PC-3 cells, but showed minimal effect on the non-cancerous PNT1A cells. Accordingly, a concentration of 0.01× GMI™ was selected for the experimentation described in the following examples. This concentration is representative of the dose of the GMI™ preparation used in the case history treatments described in Example 2.

### Example 5

### Evaluation of Treatments (III)

### Materials and Methods

Cells

This example used the same PC-3 human prostate cancer cell line and human immortalised prostate PNT1A cells (control) as that used in Example 4.

GMI Treatment

Cells were treated with a 0.01× GMI™ preparation. For cells that were also subjected to UHF radiation, the cells were treated with the GMI™ preparation approximately five minutes before the exposure to the UHF radiation commenced.

Radiowave Exposure

The cells, in a 5 mm deep medium, were given a single treatment with 434 MHz (600 W) emitted from a loop antenna at a distance of 50 mm. The single treatment comprised 2×10 minute exposures (1 minute off). The cells were maintained at 1% oxygen and cultured for three days after the exposures. Some cell samples were given no UHF exposure for control (CTRL) purposes.

Analysis

The effects of the treatment were assessed by routine cell counting and cell viability assay (ie CCK-8).

### Results and Discussion

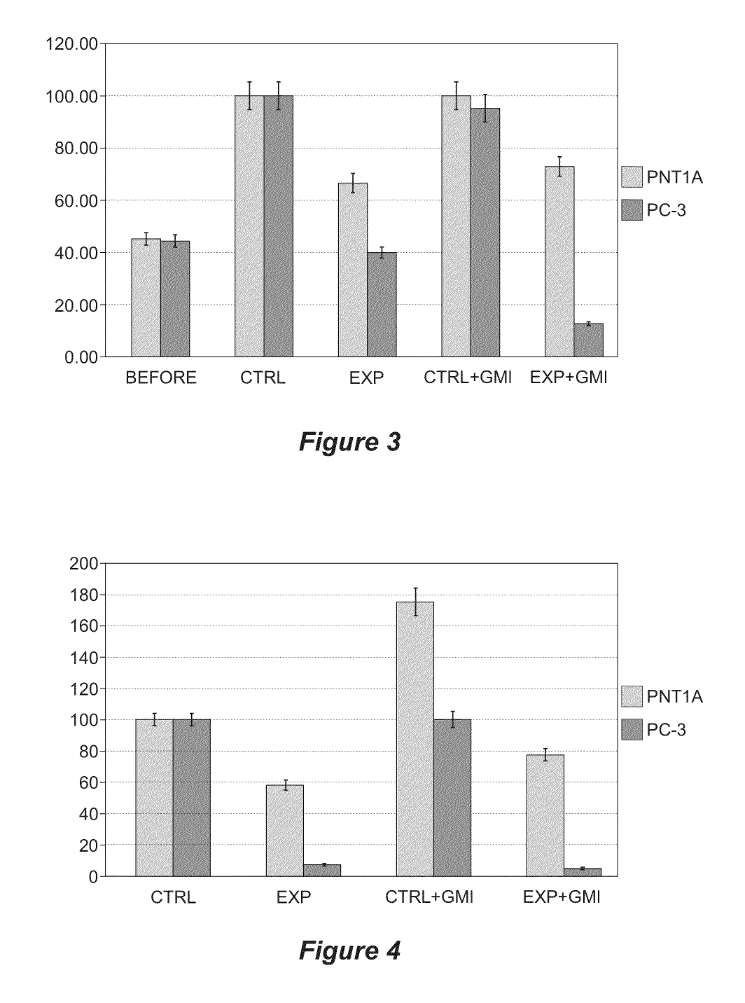

The results are shown in FIGS. 3 and 4. FIG. 3 shows relative cell counts before and after treatments (CTRL=no UHF exposure). The “before” measure indicates the cell numbers before any GMI™ treatment and/or UHF exposure. The other cell numbers shown were counted three days later. The results show that cell death has occurred in PC-3 samples exposed to UHF radiation, and GMI™ treatment followed by UHF radiation. Further, the results show that both the cancerous and non-cancerous cells are adversely affected by UHF radiation. However, notably, for non-cancerous cells that have received both GMI™ treatment and UHF radiation, there was a small increase in the number of cells (ie relative to the PNT1A cells exposed to UHF radiation only). This surprising result indicates that the GMI™ treatment is actually protecting the non-cancerous cells from the UHF radiation, while appearing to sensitise cancerous cells to the adverse (ie cell death) effects of UHF radiation. The results shown in FIG. 4 confirm the results obtained with the cell count assay. Also, the PNT1A sample treated with GMI™ but no UHF exposure, shows a higher viability, reinforcing the apparent effect of the GMI™ treatment to protect non-cancerous cells from UHF radiation.

### Example 6

### Evaluation of Treatments (IV)

### Materials and Methods

Cells

This example used the same cell types as Example 5.

GMI Treatment

Cells were treated with a 0.01× GMI™ preparation in the same manner as described in Example 5.

Radiowave Exposure

The cells, in a 20 mm deep medium, were given a double treatment with 434 MHz (600 W) emitted from a loop antenna at a distance of 50 mm. The double treatment comprised 2×10 minute exposures (1 minute off) on day one, and 2×8 minutes (1 minute off) on day two. The cells were maintained at 1% oxygen and cultured for three days after the exposures.

Analysis

The effects of the treatment were assessed as per Example 5.

### Results and Discussion

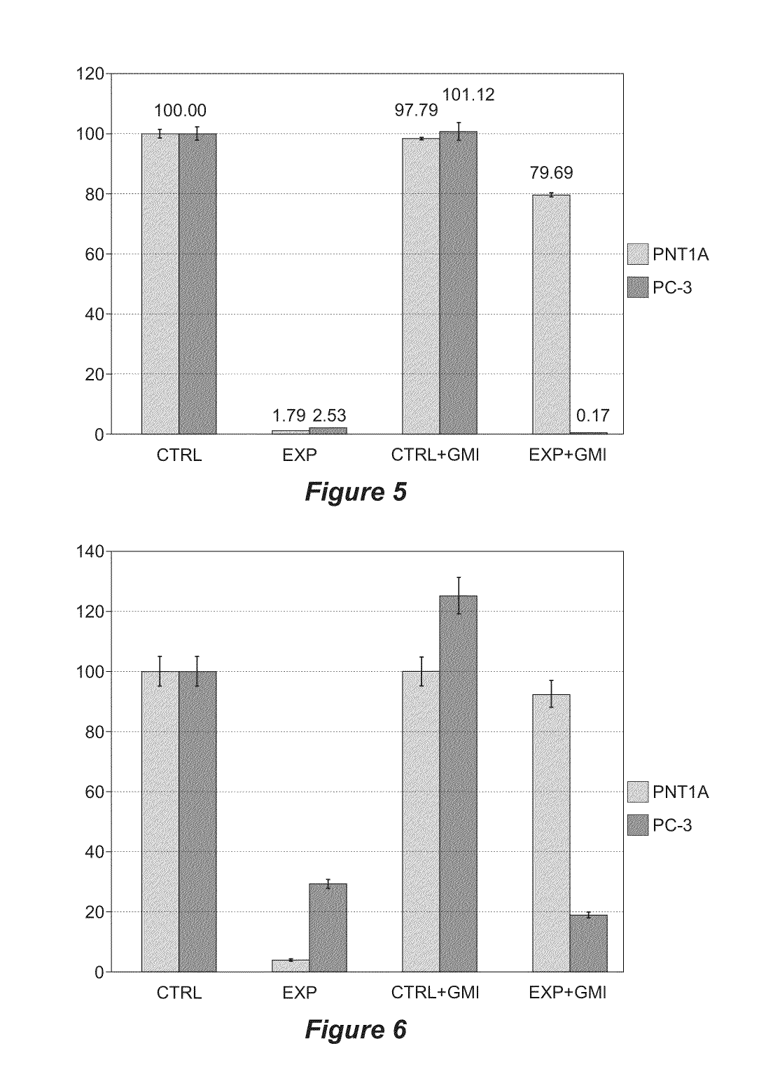

The results are shown in FIGS. 5 and 6. In particular, FIG. 5 shows relative cell counts before and after treatments (CTRL=no UHF exposure). Relative to the results obtained in Example 5, it is notable that after the two UHF treatments (EXP), the cell number of both the non-cancerous and cancerous cells is greatly decreased. When the cells are pre-treated with GMI™ but not exposed to UHF, their numbers did not vary from the control (CTRL) numbers, but when the cells are both GMI™ pre-treated and exposed to UHF, the number of the non-cancerous PNT1A cells “recovers” by up to 80%, while almost all of the cancerous PC-3 cells are dead. Since the data is expressed as a percentage of the number of cells in control flasks at the same time point, it means that, for example, the PNT1A cells post-GMI™ and UHF exposure (79.69%) have still grown in number significantly post-exposure, albeit slightly slower than control PNT1A cells. As was seen in Example 5, the cell viability assay data confirms the cell count assay results; that is, the results indicate that the GMI™ treatment protects non-cancerous cells from the deleterious effects of UHF radiation, while appearing to sensitise cancerous cells to the effects of UHF exposure.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of treating cancer in an adult subject, said method comprising an electromagnetic (EM) radiation treatment step of:
administering to the subject an effective dose of EM radiation of a frequency in the range of 430-440 MHz (UHF) within a period of ≦45 minutes, said EM radiation being provided by a UHF emitter defining an exposure region, wherein for the duration of said period the subject is reclined upon a horizontal or substantially horizontal surface, and wherein said surface and UHF emitter are arranged for relative movement such that the exposure region passes, continually or periodically, between a first position and at least a second position on said subject.

2. The method of claim 1, wherein the surface and UHF emitter are arranged for relative linear movement such that the exposure region passes, continually or periodically, back and forth between a first position and at least a second position on said subject at a rate of 5-15 feet per minute.

3. The method of claim 2, wherein the UHF emitter is in a fixed position and said surface is moved back and forth.

4. The method of claim 3, wherein the UHF emitter is an array of two or more emitters.

5. The method of claim 1, wherein the UHF emitter is an 800-1000 Watt emitter.

6. The method of claim 5, wherein the UHF emitter is positioned within 0.5 to 3 inches of the subject.

7. A method of treating cancer in an adult subject, said method comprising:
an electromagnetic (EM) radiation treatment step of administering to the subject an effective dose of EM radiation of a frequency in the range of 430-440 MHz (UHF) within a period of ≦45 minutes, said EM radiation being provided by a UHF emitter defining an exposure region, wherein for the duration of said period the subject is reclined upon a horizontal or substantially horizontal surface, and wherein said surface and UHF emitter are arranged for relative movement such that the exposure region passes, continually or periodically, between a first position and at least a second position on said subject; and
an auxiliary treatment step or steps of
(a) infusing said subject with
a dose of 3-10 g of glutathione (GSH), wherein said GSH is formulated in combination with at least one suitable oxidising agent, and/or a dose of 3-10 g of oxidised glutathione (GSSG), provided that where the subject is infused with both GSH and GSSG, the total dose of the GSH and GSSG is no more than 10 g, and
a dose of 3-10 g of S-methyl-L-cysteine sulphoxide (SMCO), wherein said SMCO is formulated in combination with at least one suitable oxidising agent,
and/or
(b) administering to said subject an effective dose of x-ray radiation.

8. The method of claim 7, wherein the surface and UHF emitter are arranged for relative linear movement such that the exposure region passes, continually or periodically, back and forth between a first position and at least a second position on said subject at a rate of 5-15 feet per minute.

9. The method of claim 7, wherein the UHF emitter is in a fixed position and said surface is moved back and forth.

10. The method of claim 9, wherein the UHF emitter is an array of two or more emitters.

11. The method of claim 7, wherein the UHF emitter is an 800-1000 Watt emitter.

12. The method of claim 11, wherein the UHF emitter is positioned within 0.5 to 3 inches of the subject.

13. The method of claim 7, wherein the auxiliary treatment step comprises infusing said subject with a dose of about 5 g of GSSG and a dose of about 5 g of SMCO.

14. The method of claim 13, wherein the EM radiation treatment step is completed within 30 minutes of the infusion of said GSSG and SMCO.

15. The method of claim 14, wherein the effective dose of EM radiation administered in the EM radiation treatment step is provided during two to four periods within the said 30 minutes.

16. The method of claim 15, wherein the said periods total about 20 to 22 minutes.

17. The method of claim 15, wherein during one of said periods, the effective dose of EM radiation is administered across the subject's whole body.

18. The method of claim 7, wherein the auxiliary treatment step and the EM radiation treatment step are both repeated on at least nine separate days thereafter within 3 weeks of commencement of the method.

19. The method of claim 18, wherein the auxiliary treatment step and the EM radiation treatment step are both repeated on fifteen separate days within said 3 weeks.
